(19) United States
(12) Patent Application Publication
(10) Pub. No.: US 2026/0283607 A1
(43) Pub. Date: Sep. 24, 2026

(54) A DELIVERY SHEATH FOR A VASCULAR CLOSURE DEVICE AND/OR A VASCULAR CLOSURE DEVICE

(71) Applicant: MOR RESEARCH APPLICATIONS LTD., Ramat Gan (IL)

(72) Inventors: Ronen JAFFE, YA'AD (IL); Eytan JAFFE, Alon Hagalil (IL)

(21) Appl. No.: 19/476,695

(22) PCT Filed: Apr. 18, 2024
(86) PCT No.: PCT/IB2024/053789

§ 371 Date: Oct. 19, 2025

(30) Foreign Application Priority Data
Apr. 20, 2023 (IL) 302311

(51) Int. Cl. A61B 17/00; A61B 90/00; A61M 25/00; A61M 25/06

(52) U.S. Cl. CPC A61B 17/0057; A61B 90/39; A61M 25/0045; A61M 25/005; A61M 25/007; A61M 25/0074; A61M 25/0668; A61B 2017/0061; A61B 2017/00623; A61B 2017/00778; A61B 2017/00955; A61B 2090/3937; A61M 2205/0216; A61M 2210/12

(57) ABSTRACT

A delivery sheath used for deploying a closure device in a blood vessel includes a cylindrical wall that surrounds a passageway. The delivery sheath further includes a side window formed through the peripheral wall at a location in between its proximal and distal ends for allowing deployment of a closure device into the blood vessel through the delivery sheath.

### TECHNICAL FIELD

Embodiments of the invention relate to a delivery sheath for a vascular closure device and/or a vascular closure device, in particular for mitigating access site bleeding.

### BACKGROUND

Access site bleeding can occur at a site where a catheter or other medical devices were inserted into a blood vessel. This can happen during or after a medical procedure such as a catheterization or angiography.

During a minimally invasive procedure, e.g., a guidewire can be inserted through the skin and into the blood vessel, and a catheter or other devices can advance along the guidewire into the blood vessel. Once the procedure is completed, the guidewire and catheter are removed, and pressure is applied to the access site to help stop bleeding.

In some cases, bleeding can occur from the access site despite efforts to control it. Access site bleeding can range from mild to severe, and can lead to complications such as hematoma (a localized swelling filled with blood), pseudoaneurysm (a bulge in the artery caused by bleeding), or even life-threatening hemorrhage.

Transfemoral TAVR (Transcatheter Aortic Valve Replacement) is one example of a minimally invasive procedure that can result in relatively severe access site bleeding due to the relatively large bore size of the device that is required in order to access the blood vessel. Transfemoral TAVR involves inserting a replacement valve e.g. through an incision in the groin and threading it up through the femoral artery towards the heart.

To reduce the risk of access site bleeding, physicians typically use specialized closure devices to seal the hole in the blood vessel wall.

Vascular closure devices can also be used for closing relative large arterial puncture sites following a catheterization procedure. Measuring the distance from the skin surface to the blood vessel may assist in deploying the vascular closure device.

### SUMMARY

The following embodiments and aspects thereof are described and illustrated in conjunction with systems, tools and methods which are meant to be exemplary and illustrative, not limiting in scope.

In an embodiment there is provided a delivery sheath for deploying a closure device into a blood vessel, the delivery sheath having a generally cylindrical peripheral wall that extends between proximal and distal ends and surrounds a passageway, wherein the delivery sheath further comprising a side window formed through the peripheral wall at a location in between the proximal and distal ends for communicating between the passageway and the exterior of the sheath.

The distal and proximal are defined relative to human anatomical plane once the delivery sheath is “operably engaged with” the patient body (e.g., inferior or superior pointing directions).

At least in certain cases, when the delivery sheath is deployed within a blood vessel, the side window may be located along an outermost boundary of a bent section of the delivery sheath. This bent section may gradually transition between a first (possibly generally straight) proximal section of the delivery sheath that extends along the path that the sheath advanced towards the blood vessel and a second (possibly generally straight) distal section of the sheath that extends along the blood vessel.

The shape of the side window can have an impact on the bending of the delivery sheath and the resulting shape of the bend. In certain embodiments, a boundary of the side window may be generally symmetrical, such as circular or elliptical, so that the bending may be more evenly and smooth around the opening. In certain embodiments, smooth transitions or curves may be designed into relative sharp corners in the boundary of the side window to avoid buckling or collapse of the delivery sheath.

In certain embodiments, once a stent-like device of the closure device at least partially protrudes out of the side window into the blood vessel, an action of pulling and retrieving the delivery sheath out of the blood vessel in the proximal direction may be performed resulting in the distal side of the side window initially engaging against the stent-like device that is maintained in its location within the blood vessel via a push arm of the closure device that is detachably attached to the stent-like device at its distal region.

During the retrieval of the delivery sheath, the stent-like device may act as a blade like member that cuts or shoves itself through a separation line of the sheath that extends from the distal side of the side window to the distal end of the sheath.

The push arm of the closure device can be designed to resist twisting about its axial extension in order to maintain the stent-like device at the suitable side to be maneuvered through the side window towards the blood vessel. Improved resistance to twisting may be accomplished by designing the push arm to have a circular cross section. Choice of a material for the push arm that has a relative high shear modulus, such as steel, may also improve in certain embodiments the push arm's resistance to twisting. Finally, in certain embodiments incorporating features such as ribs or flutes along the length of the push arm can also increase resistance to twisting in certain push arm embodiments.

In certain embodiments, once a stent-like device of the closure device is maneuvered to substantially fully protrude out of the side window into the blood vessel, an action of pulling and retrieving the delivery sheath out of the blood vessel in the proximal direction may be performed resulting in the distal side of the side window being guided to slide along an inner lateral side of the push arm that is opposite to the lateral side of the push arm from which the stent-like device protrudes away.

In certain embodiments, a boundary of the side window may be designed to encourage such sliding along the inner lateral side of the push arm. In certain cases, the boundary of the side window may be designed to have an elliptical shape with a major axis of the ellipse being generally aligned alongside the sheath's longitudinal axis. Alternatively or in addition, a distal side of the side window may be designed to bulge slightly inwards into the internal passageway of the delivery sheath in order to fit over the inner lateral side of the push arm.

During the retrieval of the delivery sheath, the stent-like device may act as a blade like member that cuts or shoves itself through a separation line of the sheath that extends from the distal side of the side window to the distal end of the sheath.

Further aspects of the present invention are exemplified in the following:
1. A method for mitigating access side bleeding in a blood vessel comprising the steps of:
   providing a delivery sheath having a generally cylindrical peripheral wall that extends between proximal and distal ends of the delivery sheath and surrounds a passageway, wherein the delivery sheath further comprising a side window formed through the peripheral wall at a location in between the proximal and distal ends for communicating between the passageway and an exterior of the sheath, and
   providing a closure device,
   advancing the delivery sheath into the blood vessel until the side window is within the blood vessel, and then
   advancing the closure device through the passageway of the delivery sheath and then out through the side window to be deployed in the blood vessel.
2. The method of aspect 1, wherein advancing the closure device through the side window comprises first advancing the closure device to a location distal to at least part of the side window and then retrieving the closure device back in the proximal direction to at least partially project into the blood vessel through the side window.
3. The method of aspects 1 and 2, wherein the delivery sheath comprises a diverter proximal to the side window for assisting in diverting the closure device through the side window as the closure device is retrieved back in the proximal direction.
4. The method of any one of aspects 1 to 3, wherein the closure device comprises a push arm and a stent-like device attached to a tip region of the push arm.
5. The method of aspect 4, wherein advancing the closure device through the passageway of the delivery sheath is via the push arm.
6 The method of aspect 5 or 6, wherein the stent-like device is detachably attached to the push arm.
7. The method of any one of aspects 4 to 6, wherein deploying the closure device in the blood vessel to mitigate access site bleeding comprises detaching the stent-like device from the push arm and activating the stent-like device to expand.
8. The method of aspect 7, wherein prior to deploying the closure device in the blood vessel to mitigate access site bleeding, the delivery sheath is removed from the blood vessel.
9. The method of aspect 8, wherein removing the delivery sheath from the blood vessel comprises engagement between a distal side of the side window and the stent-like device.
10. The method of aspect 9, wherein engagement between a distal side of the side window and the stent-like device is arranged to cut through a separation line formed in the peripheral wall of the delivery sheath between the distal side of the side window and the distal end of the delivery sheath.
11. The method of aspect 10, wherein the separation line is formed as a series of perforations through the peripheral wall.
12. The method of aspect 10, wherein the separation line is formed as slit through the peripheral wall.
13. The method of aspect 10, wherein the separation line is formed as a region of overlap between overlap regions of the peripheral wall.

In addition to the exemplary aspects and embodiments described above, further aspects and embodiments will become apparent by reference to the figures and by study of the following detailed descriptions.

### BRIEF DESCRIPTION OF THE FIGURES

Exemplary embodiments are illustrated in referenced figures. It is intended that the embodiments and figures disclosed herein are to be considered illustrative, rather than restrictive. The invention, however, both as to organization and method of operation, together with objects, features, and advantages thereof, may best be understood by reference to the following detailed description when read with the accompanying figures, in which:

FIG. 1 schematically shows a simplified view of a human circulatory system;

FIG. 2 schematically shows a delivery sheath in accordance with an embodiment of the present invention;

FIGS. 3A to 3C schematically show various stages of deploying a delivery sheath in accordance with various embodiments of the present invention into a blood vessel;

FIGS. 4A to 4E schematically show various stages of deploying an embodiment of a closure device into a blood vessel through a delivery sheath such as that seen in FIGS. 3A to 3C;

FIGS. 5A to 5G schematically show various stages of deploying another embodiment of a closure device into a blood vessel through a delivery sheath such as that seen in FIGS. 3A to 3C;

FIG. 6 schematically shows a cross sectional view of a delivery sheath in accordance with an embodiment of the present invention;

FIGS. 7A, 7B, 8A and 8B schematically show examples of diverter mechanisms possibly included in various delivery sheath embodiments for diverting into a blood vessel stent-like devices in accordance with various embodiments of the present invention;

FIG. 9 schematically shows another embodiment of a diverter mechanism in accordance with the present disclosure; and

FIG. 10 schematically shows another embodiment of a stent-like device.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated within the figures to indicate like elements.

### DETAILED DESCRIPTION

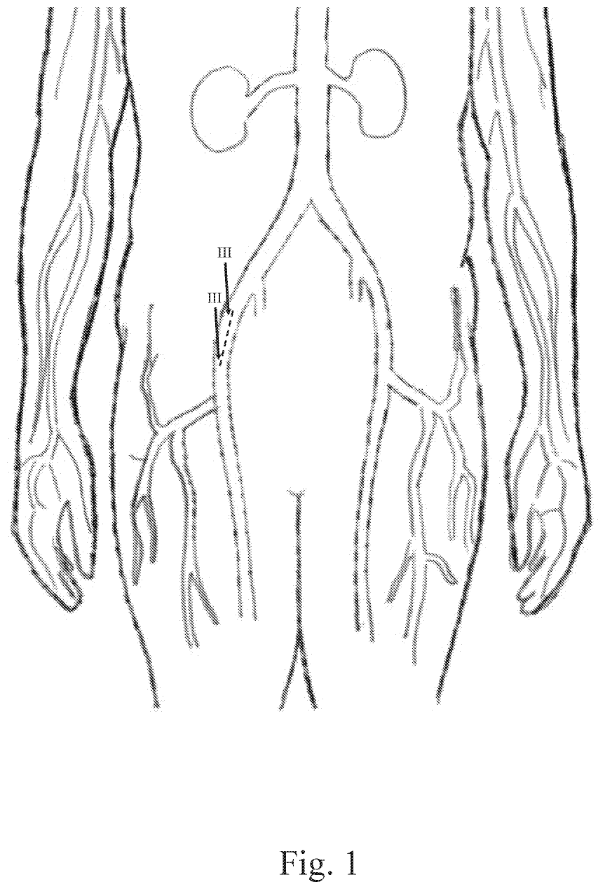

Attention is first drawn to FIG. 1 schematically showing a simplified view of a human circulatory system. Section III-III marked in this figure illustrates an access site where a relative large bore may be formed in a blood vessel, in this example the femoral artery that may serve as an access site for a transfemoral TAVR (Transcatheter Aortic Valve Replacement) procedure.

Further examples of access sites in the circulatory system where relative large bores in blood vessels may be formed, and where embodiments of the present invention may be useful in mitigating access site bleeding-can include, inter alia, the Iliac, Renal, and Carotid arteries, axillary, subclavian and carotid arteries and femoral, axillary, subclavian and jugular veins.

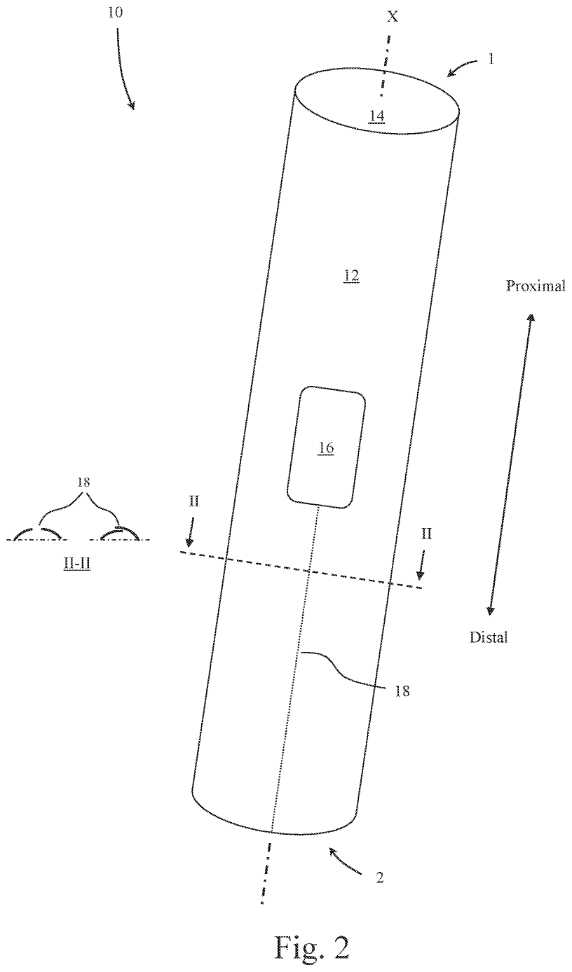

Attention is drawn to FIG. 2 schematically showing a delivery sheath 10 in accordance with an embodiment of the present invention. The delivery sheath can be seen having a generally cylindrical formation that extends along a longitudinal axis X of the sheath, with a peripheral wall 12 that is formed about an axially extending hollow passageway 14 and hence about axis X.

Passageway 14 extends between opposing axial proximal 1 and distal 2 ends of the delivery sheath. The proximal end 1 of the delivery sheath is open to provide an entry into hollow passageway 14 and the distal end 2 of the delivery sheath may be either open or closed. A hemostatic valve (not shown) can be placed at the proximal end of the delivery sheath to prevent blood from flowing back through the delivery sheath when its distal region is placed in a blood vessel. The hemostatic valve contains a one-way valve that allows fluids and devices to be infused or inserted into the blood vessel.

Delivery sheath 10 includes in addition a side window 16 that is formed through the peripheral wall 12 in between the proximal and distal ends 1, 2 of the delivery sheath. Side window 16 communicates between hollow passageway 14 and the exterior of the delivery sheath providing a sideways opening away from passageway 14 in a direction away from axis X.

Delivery sheath 10 may include in addition an optional separation line 18 that extends between a distal side of side window 16 and the distal end 2 of the delivery sheath. The peripheral wall 12 may be designed to be relatively easily separated and/or torn apart along the separation line 18.

The optional separation line 18 may be embodied in various forms. In one example it may be embodied as a perforated line weakening the material (possibly plastic type material) of the sheath's peripheral wall 12 so that the peripheral wall may be relatively easily separated and/or torn along line 18 when force is applied e.g. in a distal direction along line 18 from within side window 16. In another example, as seen in the cross sectional views II-II provided in FIG. 2, the optional separation line 18 may be embodied as an elongated slit (see left cross section) or as an overlap between sections of the sheath's peripheral wall 12 (see right cross section).

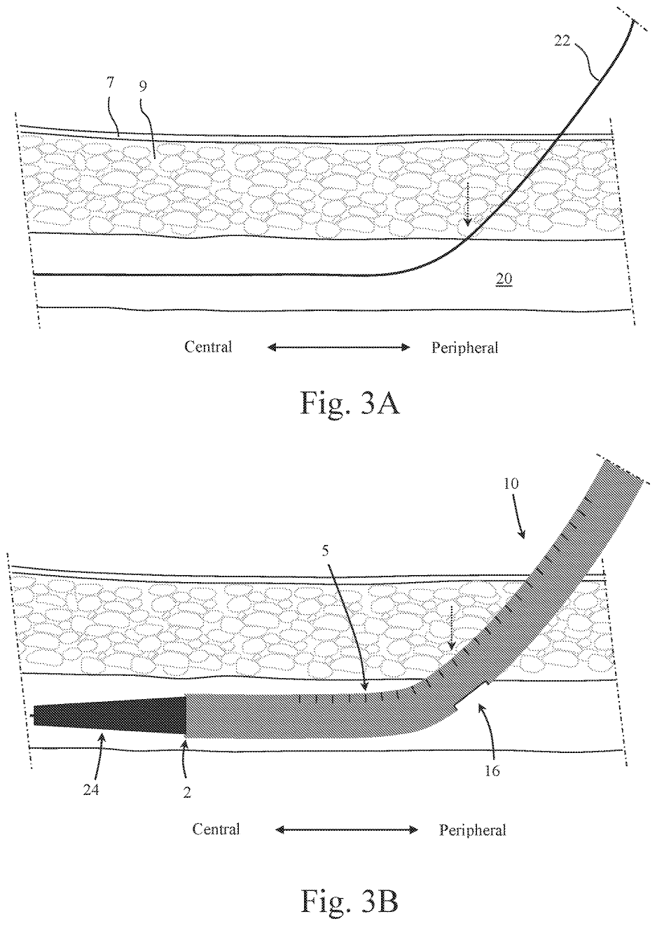

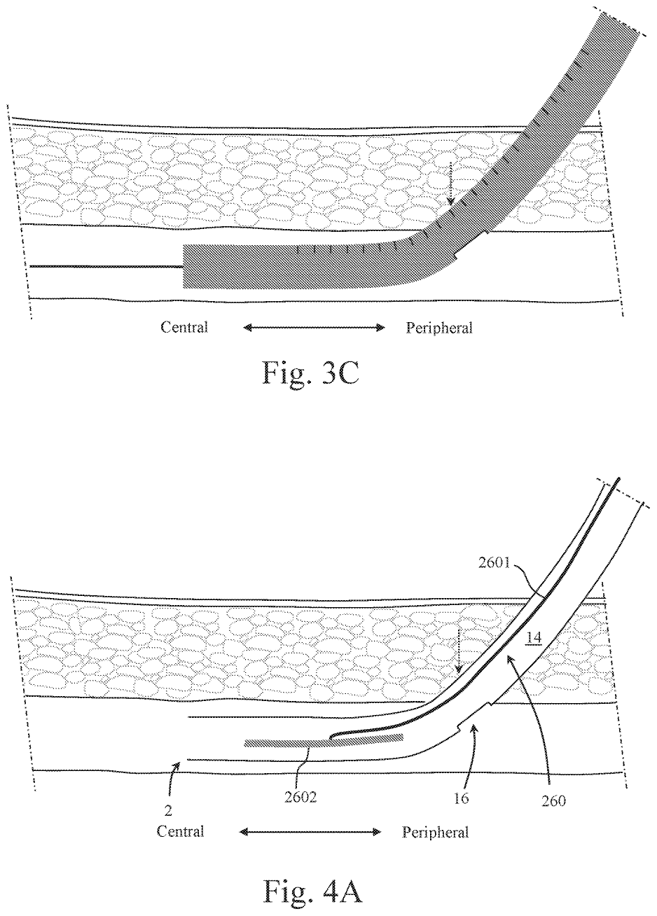

Attention is drawn to FIGS. 3A to 3C schematically showing various stages of deploying a delivery sheath, such as delivery sheath 10 that is shown in FIG. 2, into a blood vessel 20.

In FIG. 3A, a guidewire 22 can be seen being inserted through the skin 7 and fatty layer(s) 9 beneath the skin-into the blood vessel 20. The guidewire may be inserted via a needle (e.g. seldinger needle or the like). The guidewire may have been previously inserted into the blood vessel for a minimal invasive procedure that has been completed.

Possibly, use of the various embodiments of delivery sheaths and closure devices disclosed herein may be performed prior to the minimal invasive procedure was performed, in order to “park” or “position” the closure device in a location within the blood vessel for deployment after the completion of the minimal invasive procedure.

As indicated in the figures, general directional terms of “central” and “peripheral” with respect to blood vessels in the context of the present disclosure are defined. The “central” direction is the direction of insertion into a blood vessel of a guidewire (such as guidewire 22) along which e.g. delivery catheters may be initially advanced into the blood vessel.

This “central” direction, often referred to as the “cephalad” (upward) or “cranial” (toward the head) directions, may be defined as a direction towards the heart in an artery or vein based on the direction one would travel along the blood vessel to reach the heart. The “peripheral” direction is the opposing direction along the blood vessel.

The “central” direction in the example that it directs towards the heart would be the same as the direction of blood flow in veins, and the opposite direction of blood flow in arteries (except for the pulmonary artery and vein, which have the opposite direction of blood flow as compared to other arteries and veins). These directional terms will be used herein with respect to the various disclosed embodiments.

The ‘dotted’ arrow indicated in this figure, and additional figures of this disclosure, points generally to the middle of an access site where a catheter or other medical devices were previously inserted into a blood vessel. Bleeding through the access site can occur during or after a medical procedure, and may be relatively severe when a relatively large bore was required in order to access the blood vessel.

An example of a minimally invasive procedure that may result in the formation of a relative large bore at an access site may be the transfemoral TAVR (Transcatheter Aortic Valve Replacement) procedure. Further examples of minimally invasive procedures that may result in the formation of a relative large bore at an access site may include: endovascular aortic aneurysm repair (EVAR), thoracic endovascular aortic repair (TEVAR), mechanical circulatory support (MCS) and the like.

In the context of the present disclosure, an access site having a relatively large bore may be defined as an access site into a blood vessel that may have been formed by a medical device having an outer external diameter of, for example, at least about 5 millimeter or, for example, at least about 8 or 9 millimeter.

In FIG. 3B a delivery sheath, such as delivery sheath 10 seen in FIG. 2, is shown being advanced in the “central” direction along the guidewire into the blood vessel to a position where the side window 16 of the sheath is located within the blood vessel at a location proximal to the tunica externa of the blood vessel.

In an aspect of the present disclosure, when the delivery sheath is positioned within a blood vessel, the side window is arranged to generally face an opposing direction to its direction of insertion into the blood vessel. In this example where the delivery sheath was advanced in the “central” direction into the blood vessel, the side window is arranged to open out of the delivery sheath into the blood vessel generally in the “peripheral” direction. As a result, the side window provides access out of the delivery sheath in a direction that is generally opposite to its direction of insertion into the blood vessel, here in the “peripheral” direction.

Determining the depth of insertion of the delivery sheath 10 into the blood vessel so that the side window 16 is possibly located proximal to the tunica externa may be accomplished in various ways.

In one example, a depth from the skin surface to the arterial puncture site may possibly be obtained from the previously performed minimally invasive procedure, and visible markings 5 on the delivery sheath may then be used for guiding the depth of insertion into the blood vessel.

In another example, the delivery sheath may include or be assisted by means for measuring the point of entry into the blood vessel, such as a depth locator that may be inserted over the guidewire until blood flows at the proximal side of the locator outside of the body to indicate the depth to the point of entry into the blood vessel. Non-invasive imaging with (e.g. computed tomography, ultrasound or fluoroscopy) may also be used to measure the distance from the skin surface to the arterial puncture site.

A dilator 24 threaded through passageway 14 may be used for advancing the delivery sheath 10 into the blood vessel along the guidewire. Once the delivery sheath is suitably positioned within the blood vessel, the dilator 24 can be removed from within the blood vessel and the passageway as seen in FIG. 3C.

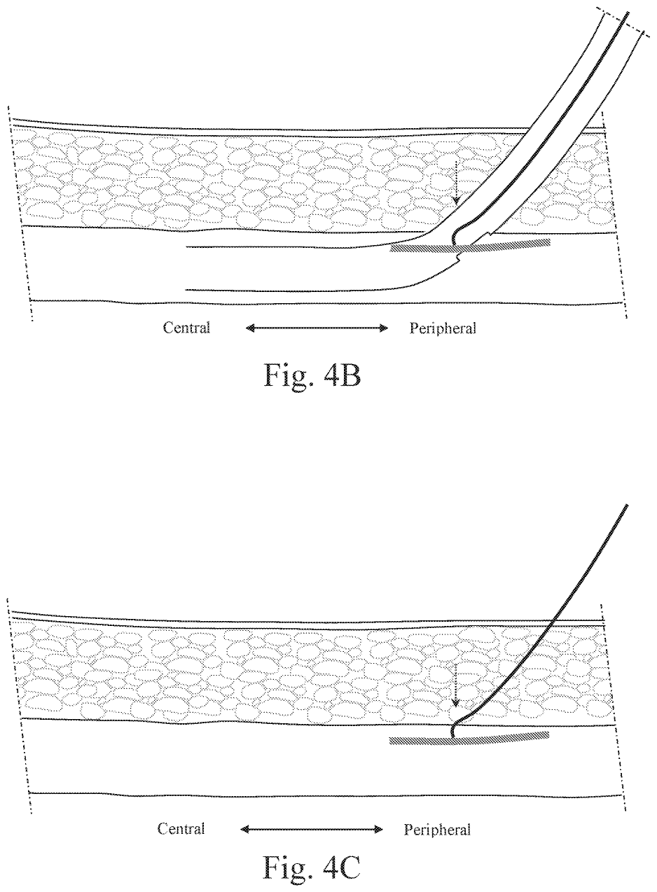

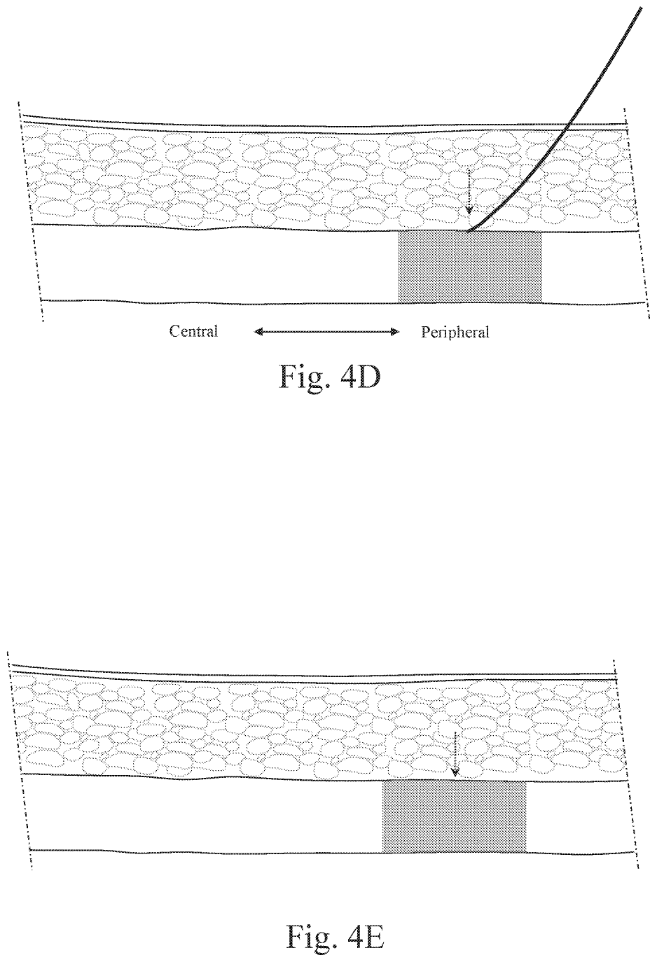

Attention is drawn to FIGS. 4A to 4E schematically showing various stages of deploying an embodiment of a closure device 260 of the present invention into the blood vessel through the delivery sheath 10. In these figures, the delivery sheath is shown in cross section to expose the closure device as it advances through the delivery sheath. Entry of the closure device into the delivery sheath may be via the hemostatic valve (not shown) that is located at the proximal side of the delivery sheath.

Closure device 260 in this example includes a push arm 2601 and an expandable nitinol-based stent-like device 2602 possibly covered with a membrane that may be bio-absorbable. The stent-like device 2602 is detachably attached in this example generally at its center to the distal end of the push arm 2601. Possibly, stent-like device 2602 is of a self-expanding type.

In the context of the present disclosure, detachment between various push arm and a stent-like embodiments may be performed in various ways. In one example, the detachment may be performed by electrolytic detachment, which is a process in which separation or detachment between the push arm and a stent-like device can be activated by using an electric current to break the chemical bond between the two objects. In such case, the electric current may be arranged to flow along the push arm towards the location where it attaches to the stent-like device.

In other embodiments, the push arm may be released from the stent-like device by applying a force that is above a pre-defined breaking limit of the attachment between the push-arm and the stent-like device. Alternatively, the push arm may be secured to the device by a screw-like mechanism and rotation of the push arm may release it from engagement with the stent-like device.

It is further noted that a stent-like device in the context of various embodiments of the present disclosure may be defined as generally relating to an expandable generally tubular/cylindrical structure.

As seen in FIG. 4A, the closure device 260 may be initially advanced via the push arm 2601 to a position within the passageway 14 of the delivery sheath where its stent-like device 2602 is located distally beyond side window 16.

From this position the closure device 260 may be retreated via the push arm 2601 back in the proximal direction until its stent-like device 2602 partially protrudes through side window 16 into the blood vessel as seen in FIG. 4B while leaving a portion of the stent-like device still within passageway 14.

As seen in this figure, in this position the center of stent-like device 2602 is generally located beneath the general location of the access site into the blood vessel, which is accordingly indicated by the ‘dotted’ arrow.

The next stage of deployment of the closure device 260 within the blood vessel may be accomplished by retreating the delivery sheath out of the blood vessel. The retrieval of the delivery sheath may involve urging the distal side of side window 16 to bear against the stent-like device 2602, which acts in its contracted state as a blade-like formation that cuts through separation line 18 in the sheath's peripheral wall 12 to thereby separate and/or tear it along this section as the delivery sheath is removed from the blood vessel.

This biasing action of the delivery sheath against the stent-like device 2602 may also assist in urging the stent-like device 2602 generally at its center to bear against the access site of the blood vessel.

Once the delivery sheath is removed from within the blood vessel as seen in FIG. 4C, the stent-like device 2602 as seen in FIGS. 4D and 4E may be activated to expand at the access site of the blood vessel while being detached from the push arm 2601.

In a broad aspect of the present invention, detachment of a stent-like device from a push arm and expansion of the stent-like device may occur in any one of the following orders: substantially simultaneously, expansion before detachment or detachment before expansion.

In the context of various embodiments of the present disclosure, a stent-like device may be enveloped in its contracted state within a constrictive wrapping that is torn away by the act of pulling away or detachment from the push-arm. Alternatively, the act of applying force upon the stent-like device in its contracted state, such as when the stent-like device acts as a blade-like formation that cuts or urges its way through the optional separation line 18, may result in the tearing of the wrapping around the stent-like device that releases it to expand.

The stent-like device when embodied having a self-expanding formation, may be inserted into a blood vessel after preparation that involves cooling and contracting it to its non-expanded state, and contact with warm blood within the blood vessel may activate shape-memory of the material (e.g. nitinol) of the stent-like device to urge it to self-expand.

In certain examples, the stent-like device may be maintained in its contracted state by synthetic woven fabric (graft), and by pulling a deployment line within the woven fabric, the stent-like device may be released to self-expand. Such a ‘deployment line’ in certain cases may be designed to extend through a lumen or passage within the push arm of the closure device, so that by pulling the ‘deployment line’ at its proximal side e.g. outside of the patient, the stent-like device may be activated to be released to self-expand.

In certain embodiments, the stent-like device may not necessarily self-expand, but rather be urged to expand by other means, such as an inflatable balloon that urges a crimped formation of the stent-like device to assume an expanded deployed state as seen e.g. in FIGS. 4D and 4E. Application of an electrical current may also be used in some embodiments to urge the stent-like device to assume its expanded shape.

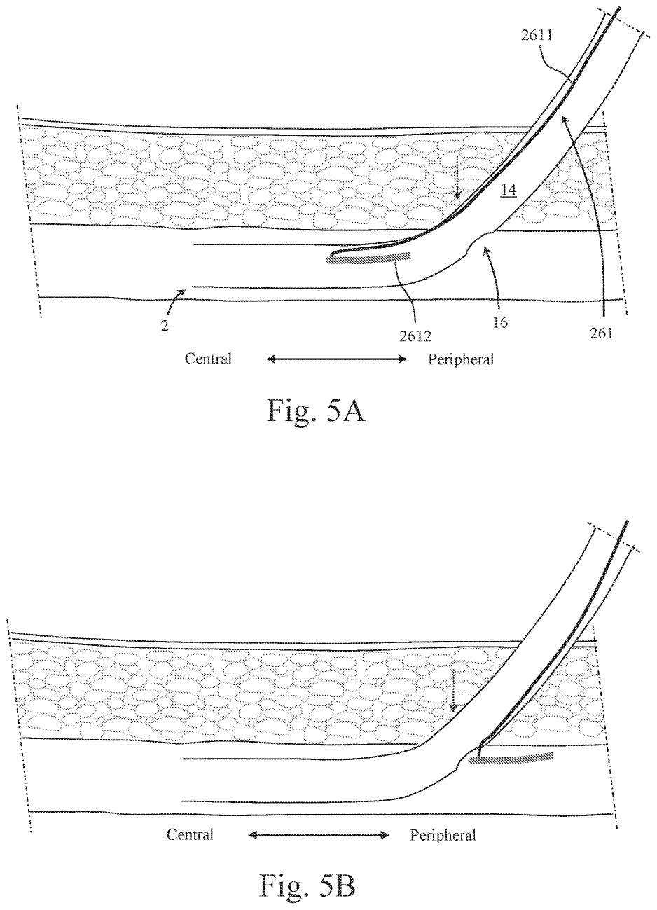

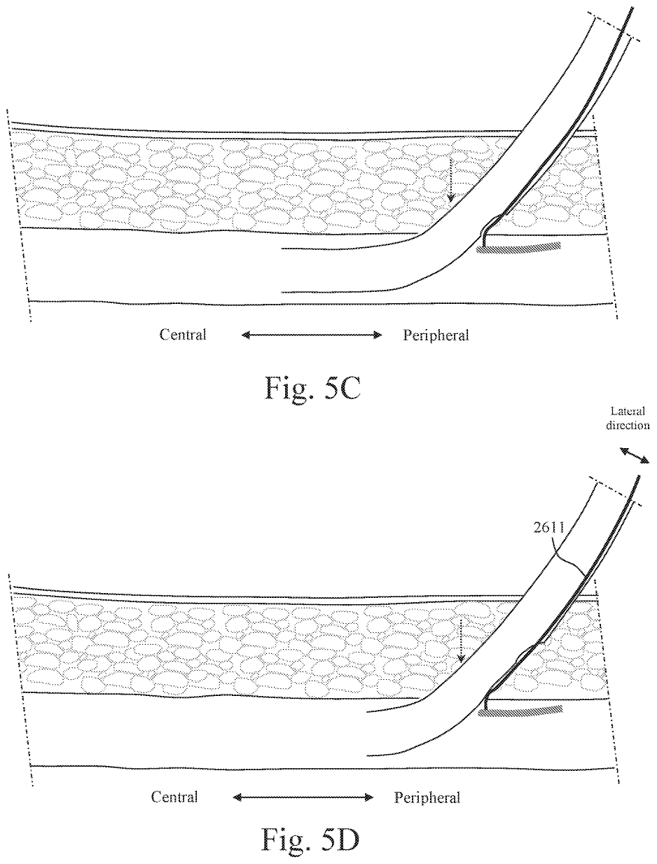

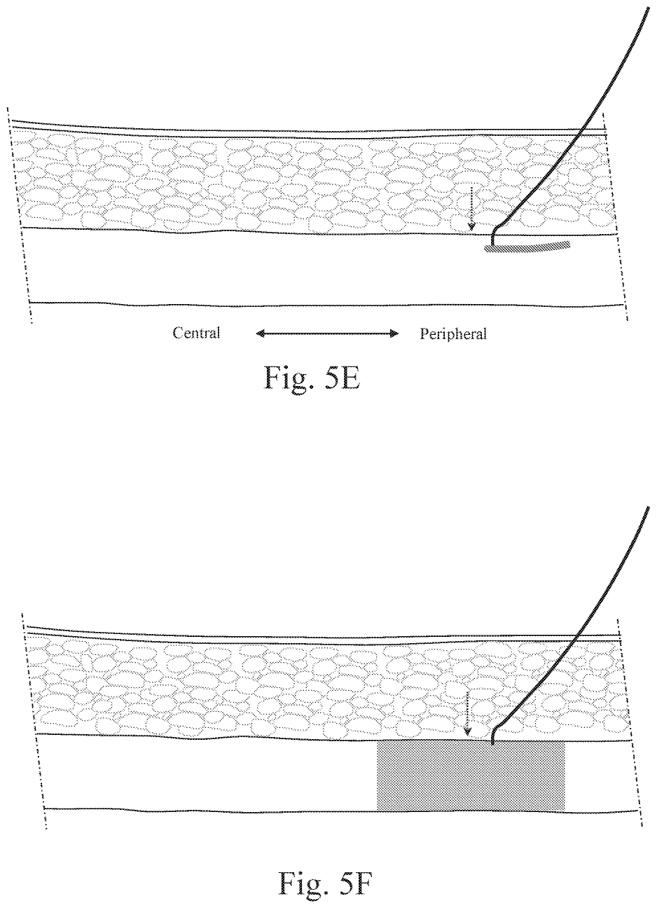

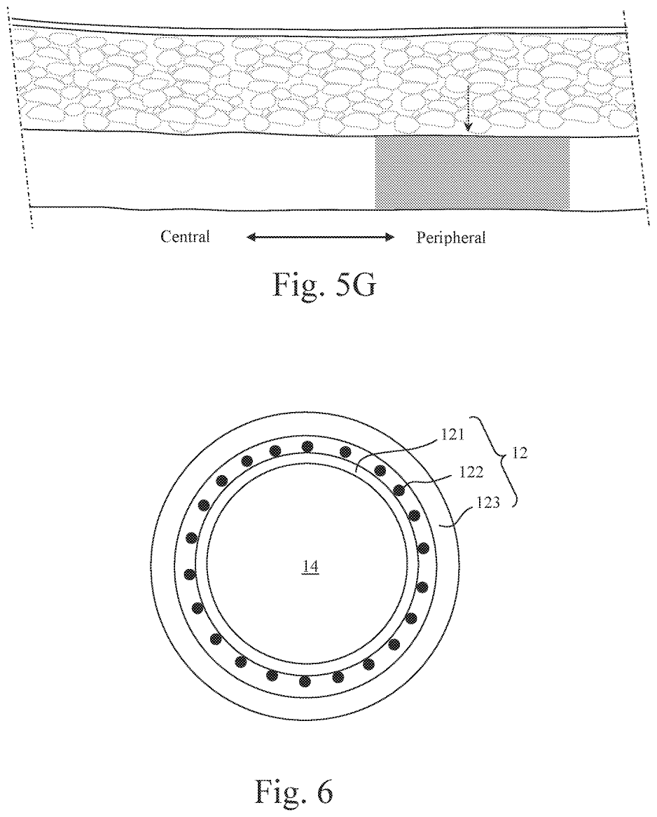

Attention is drawn to FIGS. 5A to 5F schematically showing various stages of deploying another embodiment of a closure device 261 into a blood vessel. Closure device 261 in this example includes a push arm 2611 and an expandable nitinol-based stent-like device 2612 that may be covered with a membrane, possibly a bio-absorbable membrane.

Stent-like device 2612 extends between opposing distal and proximal ends and is detachably attached in this example at its distal end to the distal end of the push arm 2611. In its attached state to the push arm and when positioned within a blood vessel, the stent-like device extends towards its proximal end in the peripheral direction of the blood vessel to form a generally V-shaped formation of the closure device 261. Possibly, stent-like device 2612 is of a self-expanding type.

As seen in FIG. 5A, the closure device 261 may be initially advanced via the push arm 2611 to a position within the passageway 14 of the delivery sheath where its stent-like device 2612 is located distally beyond side window 16.

From this position the closure device 261 may be retreated via the push arm 2611 back in the proximal direction until its stent-like device 2612 protrudes (generally in its full extension) through side window 16 into the blood vessel as seen in FIG. 5B. As seen in this figure, in this position the distal end of stent-like device 2612 is generally located beneath (or slightly shifted in the peripheral direction when positioned in an artery) with respect to the general location of the access site into the blood vessel, which is accordingly indicated by the ‘dotted’ arrow.

The next stage of deployment of the closure device 261 within the blood vessel may be accomplished by retreating the delivery sheath out of the blood vessel. The retrieval of the delivery sheath may involve urging the distal side of side window 16 to bear against the push arm 2611 of closure device 261 (at a location generally adjacent to where the push arm 2611 and stent-like device 2612 attach) in order to cut through separation line 18 in the sheath's peripheral wall 12 as already discussed herein above with respect to the previous embodiment seen in FIG. 4.

Possibly, in this embodiment the retrieval of the delivery sheath may alternatively involve urging the distal side of side window 16 to bear against the push arm 2611 of closure device 261, so that the section of sheath's peripheral wall 12 that is distal to side window 16 is now located on the opposing lateral side of the push arm 2611 as the delivery sheath is removed from the blood vessel.

In other words, as the delivery sheath is removed from the blood vessel, the section of sheath's peripheral wall 12 that is distal to side window 16 is located on a first lateral side of the push arm 2611, while the section of sheath's peripheral wall 12 that is proximal to side window 16 is located on a second opposing lateral side of the push arm 2611. This state is exemplified in FIG. 5D.

After the delivery sheath has been removed from the blood vessel as seen in FIG. 5E, stent-like device 2612 as seen is located slightly in the peripheral direction with respect to the access site of the blood vessel, which accordingly is indicated by the ‘dotted’ arrow.

In this embodiment, stent-like device 2612 when urged towards its deployed state within the blood vessel may optionally be designed while self-expanding in the radial direction to also expand in its axial direction.

One example of a self-expanding stent-like device that expands both radially and axially may be a braided stent-like device. Such an embodied braided stent-like device may include multiple wires that are woven or braided together to form a flexible mesh so that when it is deployed, the stent-like device expands radially to fit the blood vessel while also elongating in the axial direction to cover, inter alia, also the access site of the blood vessel.

In another example, the stent-like device may be designed while self-expanding to expand both radially and axially by being embodied to include a Z-stent-like device formation that is composed of multiple zigzag-shaped units that are linked together to form a flexible, expandable structure. When deployed, the Z-stent-like device expands both radially and axially in order to cover, inter alia, also the access site of the blood vessel.

In certain embodiments, stent-like device 2612 may optionally be designed while expanding radially to expand primarily in substantially only one axial direction. This may be accomplished by embodying the sent-like device to assume a “barb” or “unidirectional” formation by including a series of barbs or hooks that are oriented in a specific direction, allowing the stent-like device to be deployed to expand mainly in that direction.

In another example, stent-like device 2612 may be embodied as a “V-stent-like device”, which has a V-shaped structure that allows it to expand primarily in one axial direction.

Once the delivery sheath is removed from within the blood vessel as seen in FIG. 5E, the stent-like device 2612 as seen in FIGS. 5F and 5G may be activated to be released and self-expand in both radial and one or more axial directions in order to cover the access site of the blood vessel while being detached from the push arm 2611.

In certain embodiments, after the delivery sheath has been removed from the blood vessel and the stent-like device 2612 is located slightly in the peripheral direction with respect to the access site as seen in FIG. 5E—a n activation mechanism (not shown) located generally where the stent-like device 2612 and push arm 2611 meet—may be activated to axially move the stent-like device 2612 in it contracted state in the “central” direction with respect to the push arm's distal end. Moving the stent-like device 2612 in the “central” direction in this example may be instead of designing the stent-like device to self-expand in the radial direction while also expanding in an axial direction as previously discussed.

Such movement in the “central” direction may be controlled to position the stent-like device 2612 more centrally about the access site to the blood vessel, which is accordingly indicted by the ‘dotted’ arrow in the figures. Once generally centrally located about the blood vessel's access site, the stent-like device 2612 may be activated to be released and self-expand and detach from the push arm and said activation mechanism. The push arm and activation mechanism may then be safely removed from the blood vessel.

Attention is drawn to FIG. 6 schematically showing a cross sectional view of an embodiment of a delivery sheath of the present invention. As seen in this example, the delivery sheath's peripheral wall 12, which encloses hollow passageway 14, may be designed to include the following optional layers. An inner liner 121 possibly made from EPTFE (or the like), an optional coiled braid 122 possibly made from stainless steel (or the like), and an optional outer jacket 123 possibly made from Polyamide (Pebax) (or the like).

In various embodiments, a delivery sheath in accordance with the present disclosure may be designed along its extension to have reduced stiffness as it extends from its proximal side towards its distal side. In certain cases, the reduction in stiffness of the delivery sheath may be generally gradual and in other cases non-gradual e.g. said reduction in stiffness may change in a step wise fashion.

Reduction in stiffness of a delivery sheath may be measured in various ways, such as by Flexural Testing (subjecting the sheath to bending forces and measuring the resulting deflection), Tensile Testing (subjecting sheath to tensile (stretching) forces to measure its resistance to elongation), Compression Testing (applying compressive forces to the sheath and measuring its resistance to deformation), and the like.

In various embodiments, bending of the delivery sheath may be designed to occur adjacent its side window when deployed in a blood vessel, so that the side window will open out of the delivery sheath's hollow passageway generally in the “peripheral” direction. Such design of the delivery sheath may include imparting a higher degree of stiffness to the delivery sheath in a section substantially immediately proximal to the side window with respect to the stiffness in a more distal section of the delivery sheath in which e.g., the side window is formed.

Such formation of a higher degree of stiffness may be accomplished in various ways, such as by locating a braid within the peripheral wall of the sheath (as seen in FIG. 6) in such a section that is substantially immediately proximal to the side window, while locating a weaker braid or substantially no braid at all, in the more distal section in which e.g., the side window is formed.

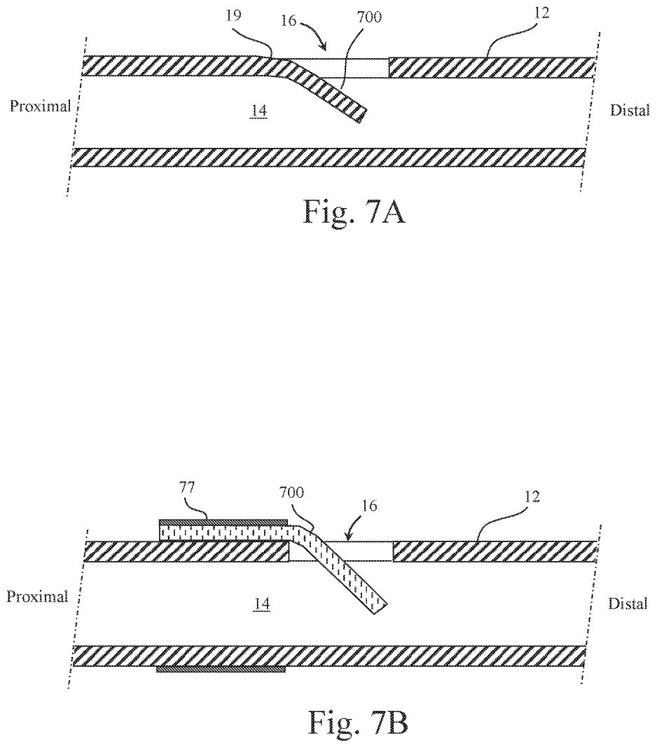

Attention is drawn to FIGS. 7A and 7B schematically showing examples of diverter mechanisms that may be included in various delivery sheath embodiments for diverting into a blood vessel stent-like devices in accordance with the various embodiments of the present invention. Such diverter mechanisms may be included in a delivery sheath to assist in diverting the stent-like devices through the respective side windows of the delivery sheaths into the blood vessels as a closure device is maneuvered in the proximal direction passed a side window.

In the cross sectional view of FIG. 7A, the diverter mechanism 700 can be seen formed as a flap like formation that was possibly cut away from the peripheral wall 12 of the sheath while remaining attached via a living hinge 19 to the remainder of the wall at the proximal side of the side window. In this example the material of the flap like formation may be designed to resiliently flex into passageway 14 in its natural non-biased state. As a result, if the flap like diverter mechanism 700 is urged away from its position within the passageway and then left alone, it will flex back to its position within the passageway,

Such resiliency of the flap like diverter mechanism may be accomplished by suitably choosing the material of the wall to be resilient and of elasticity for its intended purpose of acting as a barrier and diverter that protrudes into sheath's passageway. In one example, the portion of the wall from which the diverter mechanism 700 is formed from (or substantially all the wall of the sheath) may be formed from polymeric material, such as Polyamide, PEBAX (or the like).

After cutting away the flap like formation of the diverter mechanism, heat may be applied to the flap to relax it and then shape it into its desired form where it projects into the passageway 14 of the sheath. This process may be performed e.g. by a process of annealing that involves heating the polymeric material to a specific temperature and then allowing it to cool slowly, which helps relieve internal stresses and possibly allows the material to become more malleable.

In the cross sectional view of FIG. 7B, the diverter mechanism 700 can be seen being formed from a separate elastic member, e.g. made from nitinol, which is shaped to project into the passageway 14. Here a proximal section of the separate elastic member 700 is placed on the outer side of the sheath while a distal section thereof protrudes into the sheath's passageway. A shrink sleeve 77 made from thermoplastic material (e.g. nylon) that is placed around the section of the sheath where the proximal section elastic member is located, can be urged to conform to its shape by being exposed to heat in order to secure the elastic member 700 at its intended position along the sheath.

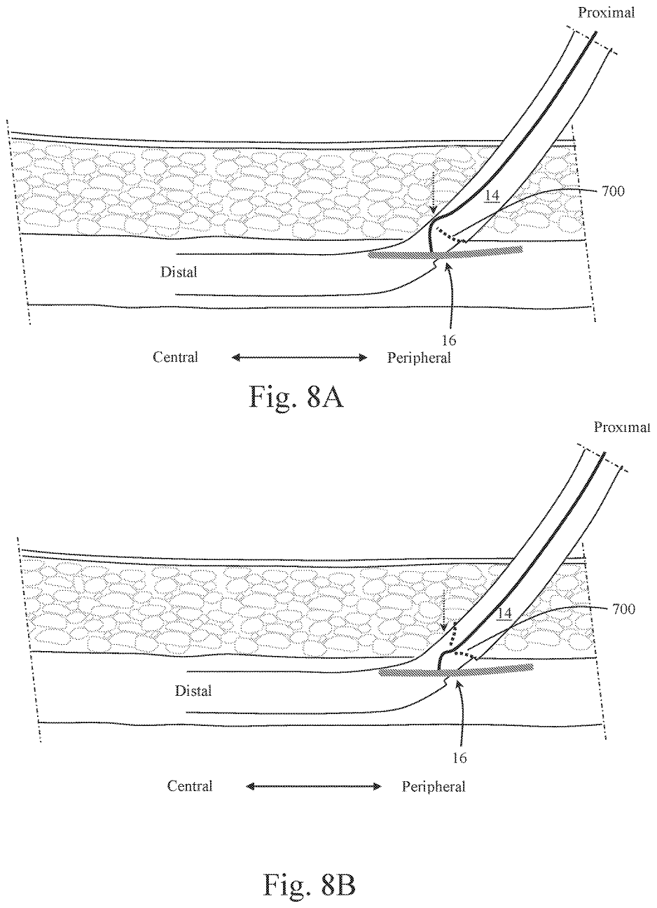

Attention is drawn to FIGS. 8A and 8B schematically showing examples of diverter mechanisms. In FIG. 8A, a diverter mechanisms 700 can be seen being positioned at the proximal side of the side window 16. In one example, diverter mechanisms 700 may be formed as a flap that was accordingly possibly cut away from the peripheral wall of the sheath while remaining attached via a living hinge to the proximal side of the side window.

Diverter mechanism 700 may be designed to resiliently flex into passageway 14. As a closure device is advanced in the distal direction and engages the diverter mechanism it will flex it towards the opening of the side window 16. Once located beyond the side window, the diverter mechanism may be substantially free to flex back into passageway 14 to act as a diverting means for assisting in diverting the stent-like device of the closure device through the side window into the blood vessel as the closure device is retrieved back in the proximal direction.

Diverter mechanisms in accordance with various embodiments of the present invention may assume various forms for assisting in diverting stent-like devices through a sheath's side window into a blood vessel. For example, a diverter mechanism 700 as seen in FIG. 8B may be designed to have a generally funnel shape that converges in the distal direction.

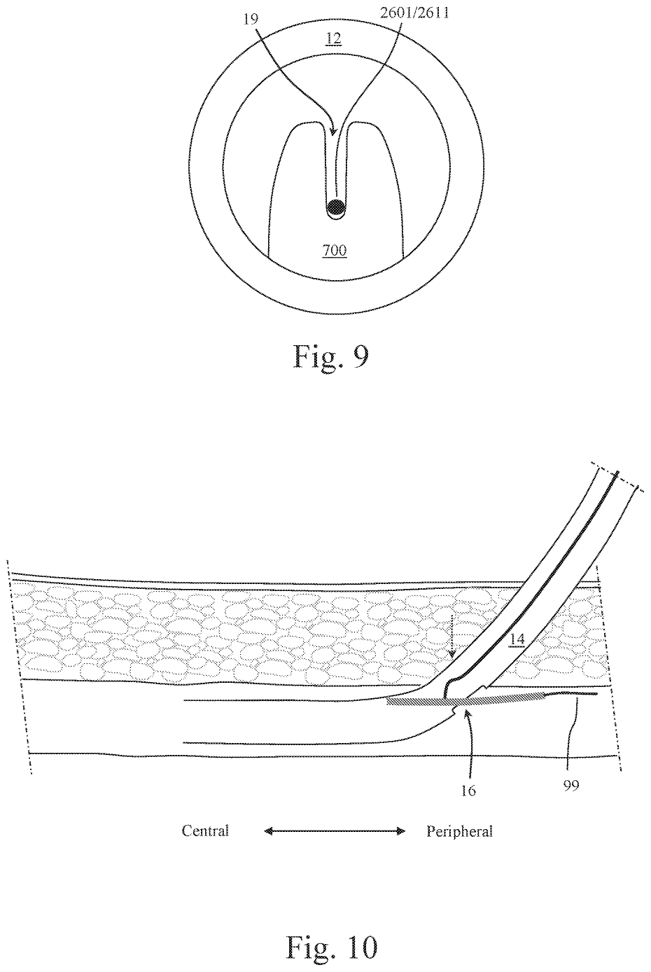

Attention is drawn to FIG. 9 showing one example of a diverter mechanism 700 when viewed from the proximal side of the delivery sheath. Here the diverter mechanism 700 can be seen including a central slit 29 that is designed to accommodate the push arm 2601, 2611 of the closure device as it extends across the diverter mechanism.

Providing space for the push arm to be accommodated within the central slit allows the diverter mechanism to protrude to a greater extent into the passageway of the delivery sheath in order to divert the stent-like device through the side window into the blood vessel as the closure device is pulled in the proximal direction passed the side window.

Attention is drawn to FIG. 10 showing another embodiment relating to a stent-like device. In order to assist in the passage of a stent-like device through the side window 16 in a non-traumatic manner with respect to the blood vessel's walls, it is possible to attach to its peripheral end a guide wire-like extension 99 (of e.g. about 0.035″ in diameter), which may assist it to navigate peripherally through the blood vessel in a non-traumatic manner.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

Further more, while the present application or technology has been illustrated and described in detail in the drawings and foregoing description, such illustration and description are to be considered illustrative or exemplary and non-restrictive; the technology is thus not limited to the disclosed embodiments. Variations to the disclosed embodiments can be understood and effected by those skilled in the art and practicing the claimed technology, from a study of the drawings, the technology, and the appended claims.

In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures can not be used to advantage.

The present technology is also understood to encompass the exact terms, features, numerical values or ranges etc., if in here such terms, features, numerical values or ranges etc. are referred to in connection with terms such as “about, ca., substantially, generally, at least” etc. In other words, “about 3” shall also comprise “3” or “substantially perpendicular” shall also comprise “perpendicular”. Any reference signs in the claims should not be considered as limiting the scope.

It is appreciated that certain features of the invention, which are, for clarity, described in the context of separate embodiments or example, may also be provided in combination in a single embodiment. Conversely, various features of the invention, which are, for brevity, described in the context of a single embodiment, example and/or option, may also be provided separately or in any suitable subcombination or as suitable in any other described embodiment, example or option of the invention. Certain features described in the context of various embodiments, examples and/or options are not to be considered essential features of those embodiments, unless the embodiment, example and/or option is inoperative without those elements.

It should be appreciated that combination of features disclosed in different embodiments may also be considered.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Although the present embodiments have been described to a certain degree of particularity, it should be understood that various alterations and modifications could be made without departing from the scope of the invention as hereinafter claimed.

1. A delivery sheath for deploying a closure device in a blood vessel, the delivery sheath having a tubular passageway confined by a peripheral wall that axially extends between proximal and distal axial ends,
wherein the delivery sheath further comprising a side window formed through the peripheral wall laterally sideways away from the delivery sheath's axis towards a given lateral side of the delivery sheath at a location in between the proximal and distal axial ends for communicating between the passageway and an exterior of the delivery sheath.

2. The delivery sheath of claim 1, wherein the peripheral wall is formed from flexible material.

3. The delivery sheath of claim 2, wherein at least certain sections of the flexible material of the sheath comprise a braid.

4. The delivery sheath of claim 3, wherein the braid is a coiled braid.

5. The delivery sheath of claim 3 or 4, wherein the braid is embedded between polymeric layers of material.

6. The delivery sheath of claim 1 or 2, wherein the proximal end of the passageway is open.

7. The delivery sheath of claim 6, wherein the distal end of the passageway is either open of closed.

8. The delivery sheath of claim 1 and comprising a separation line that is formed in the peripheral wall substantially between a distal side of the side window and the distal end of the delivery sheath.

9. The delivery sheath of claim 8, wherein the separation line is formed as a series of perforations through the peripheral wall.

10. The delivery sheath of claim 8, wherein the separation line is formed as a slit through the peripheral wall.

11. The delivery sheath of claim 8, wherein the separation line is formed as a region of overlap between overlap regions of the peripheral wall.

12. The delivery sheath of claim 1, wherein deploying the closure device in the blood vessel is through the side window.

13. The delivery sheath of claim 12, wherein the closure device comprises a push arm and a stent-like device attached to a tip of the push arm, wherein the stent-like device is initially in a contracted state forming a generally elongated shape.

14. The delivery sheath of claim 13, wherein the stent-like device is expandable, possibly self-expandable, to form a generally cylindrical formation when in an expanded state.

15. The delivery sheath of claim 13 or 14, wherein the stent-like device is detachable from the push arm.

16. The delivery sheath of claim 15, wherein the detachment is activated by electrical current.

17. The delivery sheath of claim 15, wherein the detachment is activated by applying a force that is above a pre-defined breaking limit of the attachment between the push-arm and the stent-like device.

18. The delivery sheath of claim 15, wherein the push arm is attached to a general central region of the stent-like device at least in its contracted state.

19. The delivery sheath of claim 15, wherein the push arm is attached to a general end region of the stent-like device at least in its contracted state.

20. The delivery sheath of claim 1 and comprising a diverter located proximal to the side window that is designed to project into the passageway.

21. The delivery sheath of claim 20, wherein the diverter is in form of a flap like formation that is cut away from the peripheral wall of the sheath while remaining attached to the remainder of the peripheral wall at the proximal side of the side window.

22. The delivery sheath of claim 21, wherein the diverter is made from polymeric material, possibly the same polymeric material located at least in sections of the peripheral wall that are at or adjacent the side window.

23. The delivery sheath of claim 22, wherein the diverter is designed to resiliently flex into the passageway of the delivery sheath in its natural non-biased state.

24. The delivery sheath of claim 20, wherein the diverter is formed from a separate elastic member not forming part of the sheath's peripheral wall, which is shaped to project into the passageway.

25. The delivery sheath of claim 24, wherein a portion of the diverter not projecting into the passageway and attached to the peripheral wall is attached to an outer side of the peripheral wall.

26. The delivery sheath of claim 25, wherein the attachment to the outer side of peripheral wall is via a sleeve made from thermoplastic material.

27. The delivery sheath of claim 20, wherein the diverter comprises a central slit that extends from an end region of the diverter where it generally most protrudes into the passageway towards a location where the diverter meets the peripheral wall.

28. The delivery sheath of claim 1, wherein the location where the side window is located is generally midway in between its proximal and distal axial ends.

29. The delivery sheath of claim 1 and comprising markings formed along a section of the exterior of the sheath's peripheral wall, which is generally between the side window and the proximal end of the delivery sheath.

30. The delivery sheath of claim 29, wherein the markings are formed on a lateral side of the delivery sheath that is opposite to the given lateral side and side window.

31. The delivery sheath of claim 1, wherein the delivery sheath comprises a one piece unitary construction.

32. The delivery sheath of claim 31, wherein the one piece unitary construction is of the tubular passageway of the delivery sheath.

33. A closure device for mitigating in its deployed state access site bleeding in a blood vessel and comprising in its non-deployed state a push arm and an axial extending stent-like device that is attached to an end of the push arm.

34. The closure device of claim 33, wherein the stent-like device is expandable from a contracted state where it has a generally elongated rod-like formation towards an expanded state where it has a generally tubular/cylindrical formation.

35. The closure device of claim 34, wherein prior to deployment in a state suitable for mitigating access site bleeding the stent-like device is in its contracted state.

36. The closure device of claim 35, wherein deployment of the closure device in a state suitable for mitigating access site bleeding comprises urging the stent-like device to expand and urging detachment of the stent-like device from the push arm.

37. The closure device of claim 36, wherein the detachment and expansion occurs in any one of the following orders: substantially simultaneously, expansion before detachment or detachment before expansion.

38. The closure device of claim 33, wherein in its non-deployed state the push arm is attached to the stent-like device in a location in between its axial ends.

39. The closure device of claim 33, wherein in its non-deployed state the push arm is attached to the stent-like device in a location generally adjacent one of its axial ends.

40. A method for mitigating access side bleeding in a blood vessel comprising the steps of:
providing a delivery sheath having a generally cylindrical peripheral wall that extends between proximal and distal axial ends of the delivery sheath and surrounds a passageway, wherein the delivery sheath further comprising a side window formed through the peripheral wall at a location in between the proximal and distal ends for communicating between the passageway and an exterior of the sheath, and
providing a closure device,
advancing the delivery sheath into the blood vessel until the side window is within the blood vessel, and then
advancing the closure device through the passageway of the delivery sheath and then out through the side window to be deployed in the blood vessel.

41. The method of claim 40, wherein advancing the closure device through the side window comprises first advancing the closure device to a location distal to at least part of the side window and then retrieving the closure device back in the proximal direction to at least partially project into the blood vessel through the side window.

42. The method of claim 41, wherein the delivery sheath comprises a diverter proximal to the side window for assisting in diverting the closure device through the side window as the closure device is retrieved back in the proximal direction.

43. The method of claim 42, wherein the closure device comprises a push arm and a stent-like device attached to a tip region of the push arm.

44. The method of claim 43, wherein advancing the closure device through the passageway of the delivery sheath is via the push arm.

45. The method of claim 44, wherein the stent-like device is detachably attached to the push arm.

46. The method of claim 45, wherein deploying the closure device in the blood vessel to mitigate access site bleeding comprises detaching the stent-like device from the push arm and activating the stent-like device to expand.

47. The method of claim 46, wherein prior to deploying the closure device in the blood vessel to mitigate access site bleeding, the delivery sheath is removed from the blood vessel.

48. The method of claim 47, wherein removing the delivery sheath from the blood vessel comprises engagement between a distal side of the side window and the stent-like device.

49. The method of claim 48, wherein engagement between a distal side of the side window and the stent-like device is arranged to cut through a separation line formed in the peripheral wall of the delivery sheath between the distal side of the side window and the distal end of the delivery sheath.

50. The method of claim 49, wherein the separation line is formed as a series of perforations through the peripheral wall.

51. The method of claim 49, wherein the separation line is formed as slit through the peripheral wall.

52. The method of claim 49, wherein the separation line is formed as a region of overlap between overlap regions of the peripheral wall.
